(19) United States
(12) Patent Application Publication
(10) Pub. No.: US 2009/0138059 A1
(43) Pub. Date: May 28, 2009

(54) Heart Defibrillator With Contactless ECG Sensor For Diagnostics/Effectivity Feedback

(75) Inventor: Martin Ouwerkerk, Eindhoven (NL)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS N.V., EINDHOVEN (NL)

(21) Appl. No.: 11/720,978

(22) PCT Filed: Dec. 5, 2005
(86) PCT No.: PCT/IB05/54044

§ 371 Date: Jun. 11, 2007

(30) Foreign Application Priority Data
Dec. 8, 2004 (EP) 04106391.8
Dec. 5, 2005 (IB) PCT/IB05/54044

(51) Int. Cl. A61N 1/39

(52) U.S. Cl. 607/5

(57) ABSTRACT

Heart defibrillator comprising a high-voltage power supply, a storage capacitor, and at least two electrodes, and at least one contactless biometric sensor. Since the biometric sensor does not need to be in contact with the skin of the patient, it maintains its sensing capabilities even through any regular clothing between the sensor and the body of which one or several biometric signal are to be measured. Therefore, an initial assessment of the health state of a patient can be quickly obtained. The high-voltage power supply, the storage capacitor and the at least two electrodes are used for producing an electrical pulse and applying said pulse to a patient.

The present invention relates generally to defibrillators, integrated electrocardiogram (ECG) analysis functionality, and more particularly to automated external defibrillators (AEDs).

Automated external defibrillators are generally able to monitor and analyze electrocardiogram data obtained from a patient and to determine whether the patient's ECG indicates a cardiac rhythm that may be treated with a defibrillation pulse. Based on this analysis of the patient's ECG, the rescuer, who could be a layman, is advised of initiating the defibrillation treatment.

An AED typically obtains ECG data from a patient through electrodes placed on the patient. The AED evaluates the ECG data and makes a binary shock/no-shock decision based on the ECG evaluation. The AED then reports the shock/no-shock decision to the operator of the AED and instructs him about the following steps that need to be executed.

Currently, for making an initial evaluation regarding the necessity of defibrillation to be applied to a patient, the rescuer has to place two electrodes on the chest of the patient. The electrodes need to be attached directly to the skin so that a weak electrical current defining the ECG signal can be picked up by the electrodes. This requires the rescuer to remove or at least open any clothing from or on the patient's chest. This impedes a fast evaluation of the patient's state of health and is particularly cumbersome, if the result of the initial evaluation shows that the patient does not need any defibrillation treatment, but rather a cardiopulmonary resuscitation (CPR) or another first aid action. The time lost for opening the patient's clothing is irretrievably lost. Furthermore, if there is only one AED available for several victims, the constraint of skin contact imposed by the electrodes prevents the rescuer from gaining a fast overview of the urgency for treatment of each patient. In addition, the electrodes are equipped with an adhesive coating covered with a protective film. Once applied to the chest of a patient, the adhesive coating loses some of its stickiness.

What is needed is an automated external defibrillator having the capability of measuring the ECG of a patient through his clothing.

Recent developments in the field of electric potential probes allow for a new approach to the detection of human body electrical activity. In “Electric potential probes —new directions in the remote sensing of the human body”, Measurement Science and Technology 13 (2002), 163-169, C. J. Harland, T. D. Clark, and R. J. Prance describe an electrical potential probe.

The present invention provides an apparatus and a method for quickly evaluating the necessity of delivering defibrillation action to a patient, and if so, for administering a defibrillation treatment to the patient.

In a preferred embodiment of the invention a heart defibrillator comprises a high-voltage power supply, a storage capacitor, at least two electrodes and at least one contactless biometric sensor. Since the biometric sensor does not need to be in contact with the skin of the patient, it maintains its sensing capabilities even through any regular clothing between the sensor and the body of which one or several biometric signal are to be measured. The high-voltage power supply, the storage capacitor and the at least two electrodes are used for producing an electrical pulse and applying said pulse to a patient. Accordingly, these components become important, if the analysis of the ECG signal showed that defibrillation is necessary.

In a related embodiment the heart defibrillator further comprises analyzing means connectable to the biometric sensor. The analyzing means perform(s) signal processing on the signal acquired via the biometric sensor in order to arrive at an evaluation of the state of health of the patient.

In a further embodiment the contactless biometric sensor is a capacitive sensor. A capacitive sensor is sensible to an electric field by measuring so-called displacement currents caused by variations of the electric field. However, no current needs to flow between the capacitive sensor and the measured object. Therefore, changes of the electrical potential in the vicinity of capacitive sensor results in a displacement current within the sensor, even if the space between the sensor and the place where the variation of the electrical potential took place is filled with an electrical insulator.

In a further embodiment of the present invention, the biometric sensor is comprised in the electrodes. Such an arrangement reduces the number of components that need to be handled by the rescuer. Furthermore, although the respective functions are quite different, the shape of each of the electrodes and of the biometric sensor can be chosen alike. While the electrodes need a large contact surface so that for a given current strengths the current density does not exceed a certain value within a limited region, the capacitive sensor benefits from a large surface in that it allows to produce a relatively strong displacement current.

In a further embodiment the heart defibrillator further comprises the decoupling means to decouple the biometric sensors while the storage capacitor is decharged through the electrodes. The decoupling means prevent that the high energetic current, which traverses the electrodes during the discharge of the storage capacitor, effects or damages any analyzing circuits connected to the biometric sensor.

In a further embodiment of the invention the heart defibrillator further comprises shielding means for said contactless sensor adapted to eliminate or reduce interference by the proximity of other persons while a measurement using said contactless sensor is performed. During a measurement using the contactless sensor, a healthy person that is standing too close to the patient could influence the result of the measurement. This could lead to a wrong estimation of the state of health of the patient. Such a misinterpretation can be avoided, if the biometric signal emitted by the healthy person is sufficiently shielded from the contactless sensor.

In a related embodiment of the invention, the shielding means comprise a conductive layer disposed on the backside of said contactless sensor and connected to ground. This leads to the contactless sensor having a strong directionality so that the rescuer (and any other person at the scene) may simply step out of the measuring region of the sensor, which, in the case of a conductive backside of the sensor, may be a lobe at the front of the sensor.

In a further embodiment of the present invention the electrodes comprise adhesives adapted to fix the electrodes on the skin of a patient. The adhesives are covered by a peelable protective film providing for non-contact measurement by means of said electrodes during a measurement using the contactless sensor to determine if the patient requires defibrillating intervention. Adhesive on the electrodes are useful for attaching the electrodes to the skin of the patient so that the defibrillating intervention can be performed properly. A peelable protective film prevents the adhesive from the drying out prematurely. Furthermore, while an initial measurement is performed using the contactless sensor, possibly on the appareled patient, the protective film prevents the electrodes from sticking to the clothing. Once it is determined that the patient does indeed need the defibrillating intervention, the protective film may be peeled so that a secure fixation of the electrodes on the skin is made possible.

In a further embodiment of the present invention, at least one contactless biometric sensor is part of an electrocardiographic device, integrated with the heart defibrillator. The analysis of the electrocardiogram of a patient is an efficient tool for determining whether or not a patient needs defibrillating intervention. An electrocardiogram (ECG) is an electrical recording of the heart and is used in the investigation of heart diseases. The electrical activity is related to the impulses that travel through the heart that determine the heart's rate and rhythm. The electrocardiographic device may be capable of displaying the electrocardiograms so that a trained rescuer is given additional information.

In another preferred embodiment of the present invention, a method for an automatic external defibrillator is disclosed. The automatic external defibrillator has a high-voltage power supply, a storage capacitor, at least two electrodes and at least one contactless biometric sensor. The method comprises:

performing an initial biometric measurement by means of the at least one contactless biometric sensor on the skin or the clothing of a patient;

determining a result of the biometric measurement as to if the patient requires defibrillating intervention;

executing as needed a defibrillating sequence by means of the high-voltage power supply, the storage capacitor and the at least two electrodes fixed to the skin of the patient.

The contactless biometric sensor is capable of measuring the given biometric signal regardless of whether it is placed directly on the skin or the clothing of the patient. The signal issued by the contactless biometric sensor is not profoundly influenced by the placement of the sensor, as long as it is operated within its specifications. However, a gap beneath a sensor may lead to signal corruption, which can be avoided by firmly placing the sensors on the clothing. Once the result of the biometric measurement is determined a decision is made whether or not the patient requires defibrillation. The automated external defibrillator may indicate such a result to the rescuer and instruct him to place the electrodes as required for a defibrillating intervention, i.e. on the bare skin of the chest of a patient. The automated external defibrillator may further wait for an acknowledgement of the rescuer as to the accomplishment of the electrodes' placement, in order to then continue with issuing a warning to the rescue to stand back from the patient. Eventually, the automated external defibrillator may execute a defibrillating sequence, possibly interrupted by further measurements to be performed by the contactless biometric sensor.

In a further embodiment of the present invention the electrodes are fixed to the skin of the patient by means of adhesive films on the electrodes. This ensures a large contact area of the electrodes with the skin and avoids movement of the electrodes.

In a further embodiment of the present invention the initial biometric measurement is or comprises an electrocardiographic measurement. An electrocardiogram is one of the most meaningful biometric signals concerning heart activity that can be measured non-invasively. It has the further benefit of being instantaneously available. Since the electrocardiogram signal also has a measurable distant effect, it is well suited for the application of a contactless biometric sensor.

The forgoing aspects and the advantages of this invention will become more readily appreciated as the same becomes better understood by a reference to the following detailed description, when taken in conjunction with the accompanying drawings, wherein:

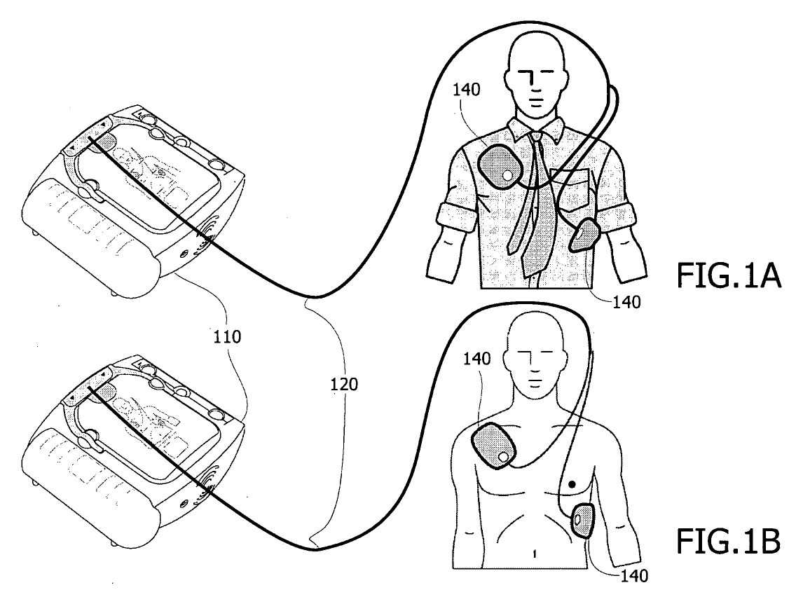

FIG. 1A depicts an automated external defibrillator and an appareled victim;

FIG. 1B depicts an automated external defibrillator and an undressed victim;

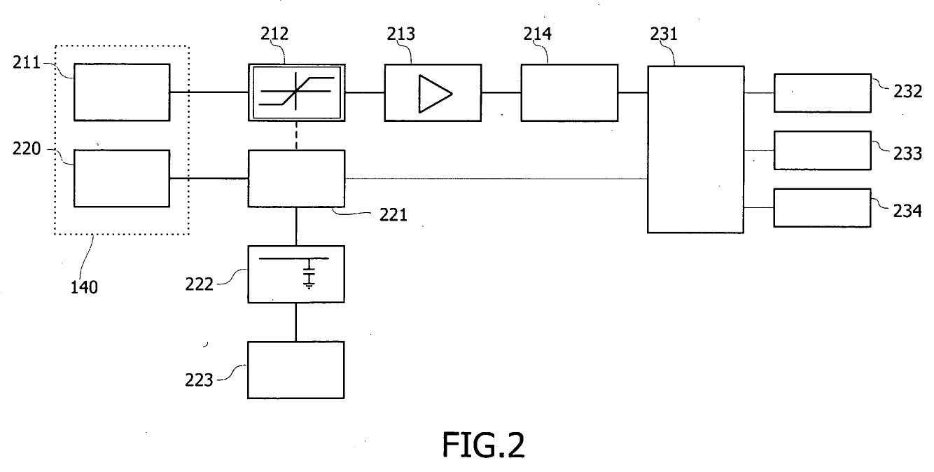

FIG. 2 is a block diagram illustrating the major components of an automated external defibrillator shown in FIG. 1A and FIG. 1B;

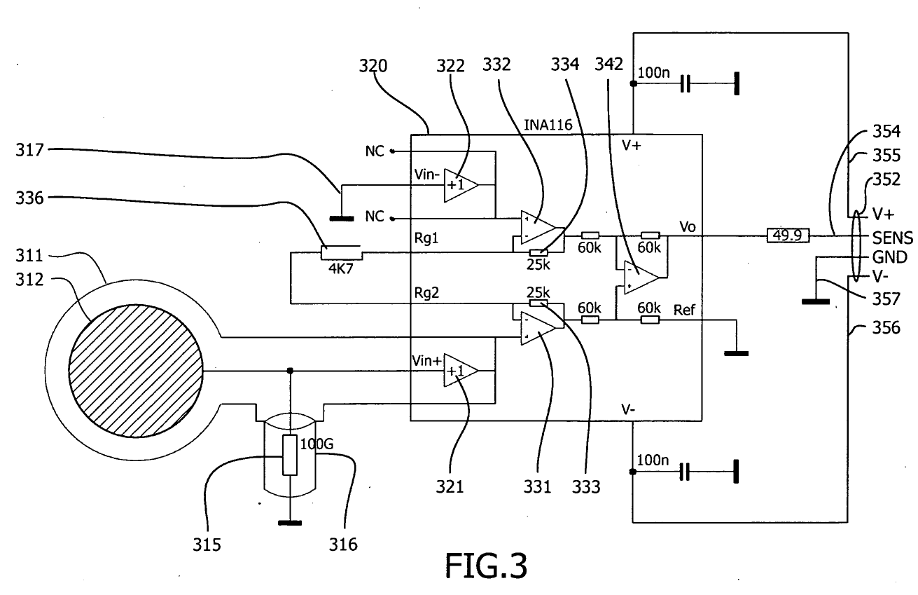

FIG. 3 shows the circuit schematics of a contactless sensor and associated amplifying circuitry; and

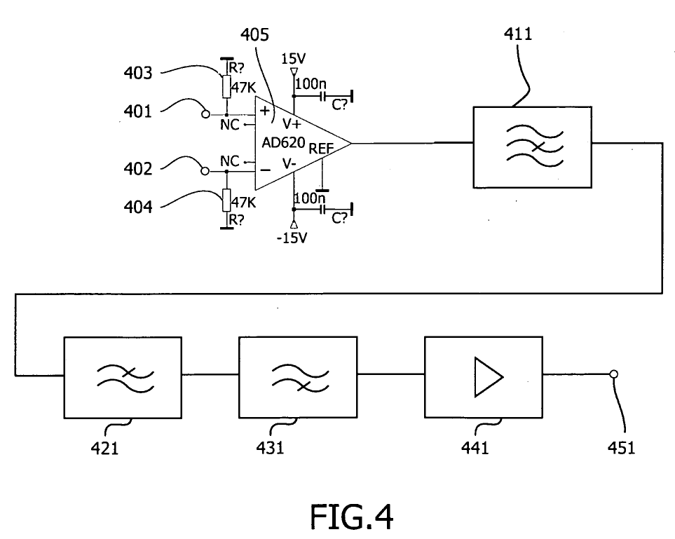

FIG. 4 shows a signal processing circuit in an automated heart defibrillator according to the present invention.

FIG. 1A shows a scenario, in which an automated external defibrillator 110 is used for measuring one or several biometric signals of a victim 105. The automated external defibrillator 110 is connected via electrodes/sensors connections 120 to two electrode/sensor housings 140. In the depicted scenario, those electrode/sensor housings 140 are placed on the clothing of the victim 105. This is made possible by contactless sensors inside the electrode/sensor housings 140, which avoid the need to place the electrode/sensor housings 140 directly on the skin of the victim 105, if, to begin with, a measurement of one or several biometric signals pertaining to the victim 105, and a subsequent evaluation of the measure biometric signal(s) is truly performed. During this stage of a first-aid intervention the automated external defibrillator 110 functions in a measurement node, in which contactless biometric sensors within the electrode/sensor housings 140 pick up biometric signals from the victim 105. These biometric signals are transmitted from the electrode/sensor housings 140 to the automated external defibrillator 110 via electrodes/sensors connections 120. Within the automated external defibrillator 110 the measured biometric signals are processed and analyzed with respect to any symptoms suggesting that a defibrillating action should be performed on the victim. Unless the victim's clothing contains materials that disturb the operation of the contactless sensors, such as large metallic objects, the contactless sensors are capable of biometric signals usually through several layers of clothing. The electrode/sensor housings 140 are immobilized with respect to the underlying skin in all directions to minimize artefacts. Artefacts in the measured biometric signals can be caused by the nature of the capacitive coupling. For example, any variation of the distance between a capacitive sensor and the skin of the victim leads to a variation of the capacitance, and consequently to a variation of a measured voltage and/or current. This ultimately deforms the measured biometric signal so that a meaningful analysis will not be possible, if the deformation becomes too strong. In the case of a capacitive sensor, this can be avoided by immobilizing the sensor with respect to the underlying skin. That can for example be achieved by using clips attached to the electrode/sensor housings 140. Another possibility would be to immobilize the electrode/sensor housings by means of a belt. Finally, the electrode/sensor housings 140 could be immobilized by placing them between the floor and the body of the victim.

FIG. 1B shows a later stage during a first-aid intervention using an automated external defibrillator 110. Prior to this stage, an analysis of measured biometric signals of the victim 105, for example during the stage of the first-aid intervention shown in FIG. 1A, revealed that it is necessary to defibrillate the victim by means of the automated external defibrillator 110. Due to the rather high current magnitude that needs to be of applied to the victim in order to achieve the desired defibrillation effect, direct contact between the electrode within the electrode/sensor housings 140 through which the current pulse flows, and the skin must be established. The rescuer is therefore required to remove the victim's clothing and to place the electrodes on the bare skin of the chest of the victim 105. As soon the rescuer has completed his/her intervention, he/she indicates to the automated external defibrillator 110, e.g. by pushing a button, that the setup required for administering a current pulse to the victim is completed. After each application of a current pulse to the victim, the response of the victim 105 is again measured and analyzed by means of the contactless sensors, in order to avoid any unnecessary and possibly harmful defibrillation. It is desired to have a large contact area between the electrodes and the skin so that a uniform distribution of the current density is achieved. Adhesives that are disposed on the electrode/sensor housing 140 provide adhesive force between the skin of the victim and the electrode/sensor housing 140. A rescuer therefore usually peels a protective film covering the adhesives and then applies the electrode/sensor housing to the skin of the victim by means of these adhesives.

FIG. 2 shows a block diagram of an automated external defibrillator 110 according the present invention. The electrode/sensor housing 140 comprises two functional components, namely a biometric sensor 211 and an electrode 220. Usually, an automatic external defibrillator is equipped with a pair of the electrode/sensor housings 140 and their components, which allows the individual placement of each of the housings 140 as a function of the body-height of the victim 105. Although the biometric sensor 211 and the electrode 220 in one of the electrode/sensor housings 140 are shown as two distinct functional elements, they can be physically integrated one with each other. The biometric sensor 211 is connectable to the decoupling means 212. The decoupling means 212 prevent(s) that any harmful voltages picked up by the biometric sensor 211 are passed on to subsequent circuits for signal processing. The decoupling means 212 can be controlled by a defibrillation circuit 221, to be described later. The biometric signal measured by the biometric sensor 211 and limited by the decoupling means 212 is then amplified in an amplifier 213. Preferably, the amplifier is or comprises an operational amplifier having a high signal-to-noise ratio. The amplified signal is then transported to analyzing means 214, connected to the amplifier 213. The analyzing means 214 tries to detect a heart rhythm in the measured biometric signal(s) and to extract characteristic parameters from that signal. The analyzing means can for example be an expert system, which has patterns of different heart rhythms stored in a memory. These patterns of heart rhythms are typical patterns, which are encountered while performing first-aid, which have been classified by medical experts during the development of these automated external defibrillator 110 and stored in the memory together with the diagnosis of the medical expert. Alternatively, or in addition, a rule-based or table-based evaluation algorithm could be implemented by the analyzing means. The analyzing means can also re-scale the biometric signal along the time axis and/or along the magnitude axis. Preferably, the analysis of the biometric signal is performed digitally, so that the analyzing means 214 may also comprise analogue-to-digital conversion means. The result of the signal analysis is passed on to a processing unit 231, which uses that result to make a determination as to whether a defibrillation should be undertaken or not. Additional information may also be passed from the analyzing means 214 to the processing means 231, such the as the biometric signal itself. The processing unit 231 is for example a micro processor or a micro controller. It is also connected to a memory 232, a display 233, and an input device 234. The memory 232 stores for example the program that is to be run by the processing unit 231 and any temporary variables or states that are generated during the executing of the program. It may furthermore, store above mentioned patterns of heart rhythms, to be loaded into the analyzing means 214. The display 233 serves as a communication means with the rescuer. The rescuer is informed about any findings of the analyzing means with respect to the measured biometric signal and further information regarding the use of the automated external defibrillator, e.g. remaining capacity of the battery, or wrong placement of the biometric sensor or the electrodes. In addition to a visual display 233, acoustic output devices may be used to instruct the rescuer acoustically, who therefore does not have to read the display frequently, but may rather listen to the acoustic instruction. The input device 234 allows the user to interact with the automated internal defibrillator. Since the placement of the sensors and the electrodes requires manual intervention of the rescuer, the automated internal defibrillator needs to be informed about the completion of such actions. Besides the components for measuring and analyzing biometric signals described above, the automated external defibrillator also comprises a high voltage circuit. In FIG. 2, the high voltage circuit comprises the electrode(s) 220, the defibrillation circuit 221, the storage capacitor 222, and a high voltage supply 223. The high voltage supply 223 charges the storage capacitor preferably on demand of either the defibrillation circuit 221 or the processing unit 231. When charged, the storage capacitor 222 contains a considerable amount of electrical charge, which can be suddenly decharged via the defibrillation circuit 221 and the electrodes 220. The defibrillation circuit 221 may influence the decharging process, e.g. by commutating the current direction, leading to the nowadays preferred bi-phasic current. The defibrillation circuit 221 can also control the decoupling means 212, e.g. by activating them, when it prepares to decharge the storage capacitor 222 through the electrodes 220.

The state of health of a human body can be revealed through the electrical (more accurately, the electromagnetic) activity of the body originating, for example, in the heart (ECG) and the brain (EEG). In conventional practice, electrical signals are detected using voltage probes in contact with the body. These probes, which have input impedances of 10⁶ to 10⁷Ω, require real charge current contact to the surface of the body, this invariably being provided by an electrolytic paste. More precisely, silver metal electrodes are applied to the skin with adhesive pads and a silver chloride gel is used to act as an electrical transducer to convert the ionic currents low in the surface of the skin into an electron flow which can then be detected by an electronic amplifier. The recently, off-body sensing of electrical activity has been achieved at room temperature with the use of a new class of sensor, the ultra-high impedance electric potential sensor. These sensors are electrometer amplifier based and combine remarkable sensibility with extremely high input impedance; sufficient in operation to allow the remote (non-contact) detection of electric potentials generated by current flowing in the body. By comparison with traditional contacting electrodes for electrical sensing, the new sensors draw only a displacement current, not a real charge current, from the body. Furthermore, with the input impedances (up to ≈10¹⁵Ω at 1 Hz) and noise levels (≈70 nV Hz^{-1/2} at 1 Hz) achievable with these sensors, non-invasive access and detection of a large number of body electrical signals of interest is now possible.

Now turning to FIG. 3, a sensor circuitry is represented. This sensor circuitry is integrated with each of the pads of an AED. Its purpose is to amplify a first signal of a sensor. In order to reduce stray pick-up of parasitic noise between the sensor and the amplification circuitry, the distance between both is kept small. The sensor comprises a probe electrode 312, which typically has a diameter of 1.5 cm to 20 cm. Further miniaturization of the electrode is projected and tests have been performed with electrodes as small as 0.5 cm in diameter. The sensor probe electrode is surrounded by a ring-shaped guard 311 and connected to the Vin+ input port of an instrumentation amplifier 320, such as the Burr-Brown INA 116. This type of instrumentation amplifier offers the option of guarding its signal input ports in a continuous manner, i.e. as well in the circuitry feeding the instrumentation amplifier 320 as within the instrumentation amplifier itself (on-chip guarding). Accordingly, the guard 311 is connected to the guard ports of the instrumentation amplifier 320 adjacent to the Vin+ input port. The connection of the probe electrode 312 and the instrumentation amplifier 320 is grounded via a leak resistor 315, which is meant to stabilize the output of the instrumentation amplifier 320 with a sufficiently fast time constant. After excessive disturbances the instrumentation amplifier 320 drifts outside its range of operation. Leak resistor 315 brings it back into range, but must not disturb the measurement signal picked up by probe electrode 312. This means that any drift compensation performed by the leak resistor must happen rather slow so that the leak resistors 315 needs to have a relatively high nominal value. Instead of using a leak resistor 315, other arrangements achieving the same effect of drift compensation are also imaginable. Leak resistor 315 is guarded by a sheath 316 shielding the electromagnetic field that is created by the leak resistor 315.

Inside the of the instrumentation amplifier 320, the measurement signal is supplied to a signal driver 321. The output of the signal driver 321 is connected to an operational amplifier 331 with negative feedback through resistor 333.

The other input port Vin−of the instrumentation amplifier 320 is connected to a ground by means of connection 317. The connection of the two input ports Vin+ and Vin− of the instrumentation amplifier 320 means that an electric field gradient will be measured and eventually cause an output of instrumentation amplifier 320. Inside the instrumentation amplifier 320 input port Vin− is connected in a similar manner as input port Vin+. The signal is first applied to a driver 322. Guarding is provided inside the instrumentation amplifier 320 by two ports adjacent to the Vin−input port and extends to the driver 322. The output of driver 322 is connected to an operational amplifier 332 with negative feedback through resistor 334. The feedback resistors 333 and 334 are mutually trimmed so that both operational amplifiers 331 and 332 have equal amplification factors. While the having trimmed feedback resistors 333 and 334 assures an equal amplification factor for both operational amplifiers 331 and 332, the actual value of the amplification factor is set by an external resistor 336 connected to the ports Rg1 and Rg2 of the instrumentation amplifier 320.

The signals amplified by each of the operational amplifiers 331 and 332 are fed to a third operational amplifier 342. In particular, the output of operational amplifier 332 is connected to the inverting input of operational amplifier 342 and the output of operational amplifier 331 is connected to the non-inverting input of operational amplifier 342. The output of operational amplifier 342 drives the output of instrumentation amplifier 320 with respect to ground potentional.

The entire sensor circuitry integrated with each of the pads of an AED is connected to the AED main unit by means of a cable 352. The cable includes a sensor signal conductor SENS 354, a positive supply voltage conductor V+ 355, a negative supply voltage conductor V− 356, and a ground potential conductor GND 357. The V+ and V− conductors are connected to ground potential via a capacitor, respectively, to assure stable supply voltage levels.

The INA 116 is shown in a configuration of a charge (Coulomb meter) amplifier, with the signal applied to the non-inverting input and the inverting input grounded. It can be seen here that, although guarding is applied to both inputs, the inverting input is treated as a dummie (i.e. grounded). The quality of the fabrication of the chip is such that the effects of low-frequency fluctuations and drift (thermally or otherwise induced) are almost exactly balanced out between the inputs. This makes the INA 116 a very suitable amplifier for the proposed purpose.

From the view point of detecting electrical activity, an ideal sensor would (1) draw no real charge current from the body, (2) have an extremely high input impedance (and thus operate as an almost perfect voltmeter), (3) have a very low noise floor, well below the smallest signal levels generated by the body, (4) be relatively low cost and (5) would appear to be perfectly biocompatible. As regards this last point, since these electric potential probes can either be used remotely or make contact to the body surface through a completely bioneutral insulating interface, biocompatibility is not a problem. Because these contactless sensors, with their remarkably high input impedance, present a negligible parallel load to the body, they are capable of fulfilling the essential point about the requirement for a perfect voltmeter. Recently, a new generation of operational amplifiers, which extends the capabilities of guarding techniques with the provision of on-chip guarding facilities has become available. An example for these operational amplifiers is the Burr-Brown INA 116 dual-input, instrumentation amplifier. A circuit design, in which such an amplifier is incorporated into a planar configured probe circuit, designed to extend the on-chip guarding to the external input electrode structure, has proved to be very successful, and a probe based on an INA 116 can be operated as an unconditionally stable charge amplifier for long periods of time. An additional advantage is that no bias current needs to be provided to the operational amplifier. Indeed, a bias current supplied to an operational amplifier leads to an unstable behavior due to the noise in the bias current path.

FIG. 4 shows a signal processing circuitry to be integrated with the main unit of the AED. Its main purpose is to improve signal quality by reducing signal to ratio and filtering of the frequency range of interest.

The signal processing circuitry has two input ports 401, 402 for each of the two sensors according to FIG. 3. Two pull-down resistors 403 and 404 assure defined voltage levels, even if input ports 401 and 402 are not connected to the respective sensors or have an indetermined voltage level for some other reason. In these situations input ports 401 and/or 402 are pulled to ground potential. An instrumentation amplifier 405 amplifies the voltage difference between the two input ports 401 and 402, corresponding to the difference of the signals measured by each of the two sensors.

The amplified differential signal is then supplied to a notch filter 411 to filter out parasitic signals of a specific frequency. Such signals are typically produced by the electricity power grid operating at for example 50 Hz in Europe and at 60 Hz in the United States. Capacitive sensors of the type used herein also measure these signals. However, given that the frequency of this parasitic signal is known and constant, a notch filter can be employed cutting out a narrow part of the spectrum that is centered around the frequency of the parasitic signal. Typical arrangements of such a notch filter include two 1^{st} order Butterworth filters.

The signal is then fed to a low pass filter 421. A typical implementation may be a Butterworth filter of first order to third order. An upper limit of the bandwidth of ECG signals at 150 Hz is commonly accepted. The application of a low-pass filter with a cut-off frequency in this range leaves the interesting spectral components of the ECG signal while filtering out obvious disturbing signals of high-frequency.

Having passed the low-pass filter 421, the signal is fed to a high-pass filter 431. Recommendations for the lower spectral bound of a ECG signal go as down as 0.3 Hz. In order to avoid that e.g. a voltage drift caused by common mode amplification of one of the operational amplifiers affects the final ECG signal, very low frequencies are filtered out by the high-pass filter 431. In addition, a limiter provides for fast DC settling.

The filter signal is once more amplified in an amplification stage 441 and is then available at output 451 for further analysis, which may be performed by a digital signal processor or a regular microprocessor.

Although the present invention has been described by means of preferred embodiments, it is not to be limited to the particular construction disclosed and/or shown in the drawings, but also comprises any modifications or variations thereto.

1. Heart defibrillator comprising a high-voltage power supply, a storage capacitor, and at least two electrodes, characterized in that it further comprises at least one contactless biometric sensor.

2. Heart defibrillator according to claim 1, further comprising analyzing means connectable to said biometric sensor.

3. Heart defibrillator according to claim 1 or 2, wherein said contactless biometric sensor is a capacitive sensor.

4. Heart defibrillator according to claims 1 to 3, wherein said contactless biometric sensor is comprised in said electrodes.

5. Heart defibrillator according to claims 1 to 4, further comprising decoupling means to decouple said biometric sensor while said storage capacitor is decharged through said electrodes.

6. Heart defibrillator according to claims 1 to 5, further comprising shielding means for said contactless sensor adapted to eliminate or reduce interference by the proximity of other persons while a measurement using said contactless sensor is performed.

7. Heart defibrillator according to claim 6, wherein said shielding means comprise a conductive layer disposed on the backside of said contactless sensor and connected to ground.

8. Heart defibrillator according to claims 1 to 7, wherein said electrodes comprise adhesives adapted to fix said electrodes on the skin of a patient and wherein said adhesives are covered by a peelable protective film providing for non-contact measurement by means of said electrodes during measurement using said biometric sensor to determine if said patient requires defibrillating intervention.

9. Heart defibrillator according to any one of claims 1 to 8, wherein said at least one biometric sensor is part of an electrocardiographic device, integrated with said heart defibrillator.

10. Method for an automatic external defibrillator, having a high-voltage power supply, a storage capacitor, at least two electrodes and at least one contactless biometric sensor, said method comprising:
performing an initial biometric measurement by means of said at least one contactless biometric sensor on the skin or the clothing of a patient;
determining a result of the biometric measurement as to if the patient requires defibrillating intervention;
executing as needed a defibrillating sequence by means of said high-voltage power supply, said storage capacitor, and said at least two electrodes fixed to the skin of the patient.

11. Method according to claim 10, wherein said electrodes are fixed to the skin of the patient by means of adhesive films on the electrodes.

12. Method according to any one of claims 10 to 11, wherein said initial biometric measurement is or comprises an electrocardiographic measurement.
